(19) United States
(12) Patent Application Publication
(10) Pub. No.: US 2009/0275497 A1
(43) Pub. Date: Nov. 5, 2009

(54) METHOD FOR IDENTIFYING MODULATORS OF THE ACTIVITY OF ION-CHANNELS RECEPTORS

(75) Inventors: Eric Boue-Grabot, Begles (FR); Jean-Rene Cazalets, Bordearx (FR); Estelle Toulme, Bordeaux (FR)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignees: CENTRE NATIONAL DE LA RECHERCHE SCIENTIFIQUE (CNRS), Paris (FR); UNIVERSITE VICTOR SEGALEN BORDEAUX 2, Bordeaux (FR)

(21) Appl. No.: 11/721,630

(22) PCT Filed: Dec. 13, 2005
(86) PCT No.: PCT/IB05/04020

§ 371 Date: Feb. 29, 2008

Related U.S. Application Data
(60) Provisional application No. 60/635,007, filed on Dec. 13, 2004.

(51) Int. Cl. A61K 38/16; C12Q 1/02; G01N 33/53; C07K 14/435

(52) U.S. Cl. 514/8; 435/29; 435/7.1; 530/395

(57) ABSTRACT

The present invention is directed to a method for selecting or identifying a compound capable of modulating the activity of an ion-channels receptor, such as GABA receptor, by measuring the calcium influx via the P2X receptor in presence of its agonist ATP when coupled to said ion-channels receptor in recombinant cells coexpressing these two receptors. The invention also concerns a kit or a device comprising the essentiel elements to perform the method according to the invention. Finally, the present invention is directed to the use of said selected compounds for the prevention or the treatment of diseases related to said ion-channels receptor dysfunction.

The present invention is directed to a method for selecting or identifying a compound capable of modulating the activity of an ion-channels receptor, such as GABA receptor, by measuring the calcium influx via the P2X receptor in presence of its agonist ATP when coupled to said ion-channels receptor in recombinant cells co-expressing these two receptors. The invention also concerns a kit or a device comprising the essentiel elements to perform the method according to the invention. Finally, the present invention is directed to the use of said selected compounds for the prevention or the treatment of diseases related to said ion-channels receptor dysfunction.

gamma-Aminobutyric acid (GABA) receptors are intrinsic membrane glycoproteins in mammal neuronal tissues that are members of the ligand-gated ion channel superfamily of receptors (named “ion-channels receptors” in the present description). GABA receptors play a major role in the inhibition of central nervous system (CNS) neuronal activity due to the widespread distribution of GABA-releasing and GABA-receptive neurons.

GABA receptors can be divided into two major classes: the GABA-A and GABA-C receptor subtypes, and GABA-B receptor types, which are distinguished by differences in their effector mechanisms and pharmacology. GABA-A and GABA-C receptors are transmitter-operated chloride channels that are activated by GABA to open their chloride channel.

GABA receptor channels, proteins which are present onto the surface of neurones, are the main inhibitory receptors of the central nervous system and are composed of five subunits that assemble to form a channel that is permeable to anions, mainly chloride ions.

There are 6 alpha subunits, 3 beta subunits, 3 gamma, 1 epsilon, 1 delta, 1 pi and 3 rho subunits. It is thought that the majority of GABA-A receptors are formed by the association of 2 alpha subunits, 2 beta and 1 gamma, although other combinations are possible. All these combinations result in receptors with different properties. All these subunits have a similar topology, with a large extracellular domain, 4 transmembrane domains and one major intracellular domain connecting the M3 and M4 transmembrane domains.

The electric currents induce by the ions movements via these ion-channels receptor complexes are low and do not allow the direct use of fluorescent probe to quantify these chloride ions influx into the cells. The method and device allowing the electrophysiological recordings of these currents are complex. For example, it is also necessary to impose a constant voltage and to work on a single cell.

So, this method using electrophysiological recordings cannot be used to perform primary screening of compounds capable of modulating such receptor channels, compound which could be of a great interest in pharmaceutical field.

The P2X receptors are ligand-gated ion channels while the other “P2 receptor”, P2Y receptors, operate generally through a G protein-coupled system. Several P2X receptor subtypes (P2X1-P2X7) form homomeric and heteromeric channels that are permeable to cations, including calcium, and which are specifically activated by extracellular ATP, its agonist. This receptor functions as a ligand-gated ion channel with relatively high calcium permeability (Egan et al., Neurosciences, 24(13):3413-3420, 2004). P2X receptors mediate membrane depolarization and Ca⁺⁺ influx and have physiological role ranging from fast excitatory synaptic transmission, pain reception to vessel vasoconstriction. Through the P2X receptors, ATP acts as an excitatory neurotransmitter in the central and peripheral nervous system.

Using electrophysiological recordings (i.e. by measuring the currents resulting from the passage of ions through the open channels), it has been already shown that GABA receptors and P2X receptors are functionally and physically coupled when they are co-expressed in a heterologous expression system (Xenopus oocytes, transfected mammalian cells or neurones expressing both receptor families) (Boué-Grabot et al., Journal of Biological Chemistry, 279:6967-6975, 2004; Boué-Grabot et al., Journal of Biological Chemistry, 279; 52519-52525, 2004).

It has been also shown that P2X interact also with nicotinic acethylcholine receptors and 5-HT3 receptors.

So, it is desirable to provide with an efficient method, no expensive and easy to perform, which can be used by high-throuhtput screening, for selecting compound capable of modulating the activity of ion-channels receptor such as GABA receptor.

This is the object of the present invention.

Surprisingly, the inventors have demonstrated that the activation of GABA-A receptors directly reduces the influx of calcium via opened P2X receptors, measured using a fluorescent calcium probe.

The inventors have also noticed by electrophysiological recordings that:
when ATP and GABA are co-applied, the observed current is lower than the sum of the individual currents, demonstrating that the two receptors exhibit reciprocal inhibition when they are co-activated. This functional inhibition is based on the molecular coupling of the receptors at their respective intracellular domains. The inventors have also noticed that that the properties of each receptor are not altered by the presence of the other type of receptor. Thus, the presence of the P2X receptor does not modify the properties of the GABA receptor with respect to its agonist (GABA). The presence of the GABA receptor does not modify the properties of the P2X receptor with respect to its agonist (ATP). Only the amplitude of the response of each receptor is decreased when they are activated simultaneously;
all the GABA receptors (GABA-A, irrespective of their molecular composition and GABA-C) inhibit the P2X2 receptors, such as P2X on co-activation;
the responses of the P2X2 receptors to ATP are stable during application (they do not desensitise) and over time (good recovery). It can therefore be used to test many compounds rapidly using one single cell; and
ATP-activated P2X receptors mediate a marked influx of calcium into a cell maintained at its resting potential.

Consequently, by co-expressing GABA and P2X receptors in a cell model, activation of the GABA receptors will be followed by modification of the fluorescence intensity of the response of the P2X receptors induced by ATP, or any of other well known P2X agonists.

So, in a first aspect, the present invention relates to a method for selecting or identifying a compound capable of modulating the activity of an ion-channels receptor, wherein said method comprises the following steps of:
a) having recombinant cells expressing said ion-channels receptor and a P2X receptor;
b) contacting said recombinant cells with the compound to be tested in presence of an agonist of said P2X receptor, preferably such as ATP, or in presence of an agonist of said P2X receptor and the agonist of said ion-channels receptor;
c) measuring the calcium influx via the P2X receptor;
d) comparing the measure obtained at step c) with the measure obtained in the same conditions for a first reference control wherein in step b) the compound to be tested is not present; and
e) selecting said compound or identifying said compound as a compound capable of modulating the activity of an ion-channels receptor, if the comparison in step d) demonstrates a significant increase or decrease of the calcium influx in the recombinant cells in presence of said compound;

If the structure of the tested or selected compound is not initially known, its structure could be identified by the structure identification methods well known by the skilled man, such as mass or U.V. spectrometry, LC-MS, sequencing, . . . after the step e) of the above method for selecting or identifying compound of the present invention.

In a preferred embodiment, the recombinant cells used in step a) do not express endogenously a receptor capable of being modulated by an agonist of said P2X. receptor, preferably such as ATP, or by an agonist of said ion-channels receptor, such as GABA if said ion-channels receptor is GABA receptor.

In a more preferred embodiment, the recombinant cells used in step a) are oocytes, which do not endogenously express receptors activated by GABA or an agonist of said P2X receptor, preferably such as ATP.

Preferably, the recombinant cells used in step a) are mammal cells or Xenopus oocytes. It is more preferable that the cell model (mammalian cells or Xenopus oocytes) co-expresses the GABA receptor of interest and the P2X receptor in a stable or transient fashion.

Preferably, said mammal cells are human, from rat or mouse.

By P2X receptor, it is intended to designate in the present description, a natural P2X receptor which has as amino acid sequence the wild type amino acid sequence of a mammal P2X receptor or a mutated mammal P2X receptor capable of interacting with said ion-channels receptor.

Among said P2X receptor, are preferred the P2X receptor selecting from the group consisting of the P2X1, P2X2, P2X3, P2X4, P2X5, P2X6 or P2X7 receptor subunit or any P2X receptor which results from an association or a combination of P2X receptor subunits capable of exercising an ATP activated channel-receptor function.

It is believed that P2X receptors comprise multimers of receptor polypeptides, which multimers may be of either the same or different subtypes. Consequently, the term “P2X receptor” refers, as appropriate, to the individual receptor subunit or subunits, as well as to the homomeric and heteromeric receptors comprised thereby.

According to a likewise particular aspect, said ion-channels receptor or said P2X receptor is from human, rat or mouse source.

The amino acid (or mRNA) sequence of these subtypes of P2X receptor (also named P2RX) are well known by the skill person and could be found, for example, in Data Bank, such as in Genbank under the following Accession Number (see Table 1):

TABLE 1
Examples of GenBank Accession Number depicted the complete sequence of P2X receptor
subtypes, or splicing variants thereof, from rat, mouse and human source.

| P2XR | | | |
|---|---|---|---|
| subtypes | Rat | Mouse | Human |
| P2X1 | X80477 | NM008771 | AF020498; U45448; AF078925 |
| P2X2 | U14414; NM_053656; | NM_153400; AB094664; | NM174873; NM174872; |
| | AF013241; AF020759; | AY044240; AB094663 | NM012226; NM170683; |
| | AF020758; AF020757; | | NM170682; NM016318 |
| | AF020756; Y10473; | | |
| | Y10474; Y10475 | | |
| P2X3 | AF084975; X91167; | NM_145526 | Y07683; AB016608 |
| | X90651; NM_031075 | | |
| P2X4 | X91200; U87270; | AF089751; AF089752; | AF012903; U83993; Y07684; |
| | U32497; X93565 | AF146516 | AF000234 |
| P2X5 | X92069; X97328 | NM_033321 | U49395; AF016709; |
| | | | AF070573; U49396 |
| P2X6 | X97376; X92070 | AB010883 | XM496501 |
| P2X7 | NM_019256 | AJ009823 | Y09561; Y12851 |

By an agonist of P2X receptor, it intended to designate in the present description, one of the P2X receptor agonist well known by the skilled person, such as ATP, α,β mATP (alphabeta-methylene ATP), benzoylbenzoic ATP (such as 2′ and 3′ mixed isomers or 2′,3′-O-(4-benzoylbenzoyl)-ATP (BzATP)), or 2-methylthio-ATP (2-MeSATP), (Zhao et al., J Pharmacol Exp Ther., 311(3):1211-7, 2004; Bianchi et al., Eur J Pharmacol. 2;376(1-2):127-38, 1999; Gendron et al., J Neurochem., 87(2):344-52, 2003), ATP being the more preferred agonist of P2X receptor.

In a preferred embodiment, said ion-channels receptor is selecting from the group consisting of the GABA receptor, the glycine receptor, the acetylcholin receptor or the serotonin receptor (5-HT3 receptor), the GABA receptor being the most preferred.

By ion-channels GABA receptor, it is intended to designate the ion-channels GABA receptor resulting from the homomeric or heteromeric association between any GABA receptor subunit, said subunit being selecting from the group of GABA receptor subunit consisting of alpha 1 to 6, beta 1 to 3, gamma 1 to 3, epsilon, delta, pi, theta, and rho 1 to 3 subunit, and said homomeric or heteromeric association forming an ion-channels receptor capable of being activated by GABA.

The amino acid (or mRNA) sequence of these GABA receptor subunits are well known by the skill person and could be found, for example, in Data Bank, such as in Genbank, under the following Accession Number (see Table 2):

TABLE 2
Examples of GenBank Accession Number depicted the complete
sequence of GABA receptor (“GABAR”) subunit, or splicing
variants thereof, from rat, mouse and human source.

| GABAR | | | |
|---|---|---|---|
| subunits | Rat | Mouse | Human |
| Alpha1 | L08490 | M86566; M57519 | X14766 |
| Alpha2 | L08491 | M86567; M57520 | S62907 |
| Alpha3 | L08492 | M86568; M57521 | S62908 |
| Alpha4 | L08493 | | U30461 |
| Alpha5 | L08494 | NM176942 | L08485 |
| Alpha6 | L08495 | X51986 | S81944 |
| Beta1 | X15466 | U14418; G755155 | X14767 |
| Beta2 | X15467 | NM008070 | S67368 |
| Beta3 | X15468 | NM008071 | M82919 |
| Gamma1 | X57514 | NM010252 | |
| Gamma2 | L08497 | M62374 | X15376 |
| Gamma3 | X63324 | X59300 | AF269144 |
| Epsilon | NM023091; AF255612 | NM017369; AF18926 | Y07637 |
| Delta | L08496 | NM008072 | AF016917 |
| theta | NM031733 | AF189260; NM020488 | AF189259; AF14468 |
| Pi | | NM146017 | U95367 |
| Rho1 | X95579; U21070 | NM008075 | M62400; M62323; |
| Rho2 | D38494 | NM008076 | M86868 |
| Rho3 | D50671 | XM484554 | |

When the ion-channels receptor is the GABA receptor, the agonist of said ion-channels receptor is GABA or any of other well known GABA receptor agonist, such as muscimol, isoguvacine.

In the method according to the present invention, the calcium influx measured at step c) is measured by a calcium probe, preferably by fluorescent calcium probe, such as calcium green, Fluo-3 or Fluo-4 probe.

In a preferred embodiment, said compound to be tested is tested for its ability to interact specifically with said P2X receptor. Said specific interacting can be demonstrated by measuring whether a significant increase or decrease of the calcium influx is obtained in the same conditions for a second reference control wherein in step a) the recombinant cells used for said second reference control express said P2X receptor and do not express said ion-channels receptor.

Particularly, said compound to be tested will be not selected if a significant increase or decrease of the calcium influx is observed in the recombinant cells for said second reference control.

In a second aspect, the present invention relates to a method for selecting or identifying an agonist of an ion-channels receptor, said method comprising the following steps of:
A) selecting a compound capable of modulating the activity of said ion-channels receptor by a method for selecting or identifying a compound capable of modulating the activity of an ion-channels receptor according to the present invention wherein in step b) said recombinant cells are contacted with the compound to be tested only in presence of an agonist of said P2X receptor, such as ATP; and
B) the selection of said compound as an agonist if the comparison carried out in step d) of the method according to the present invention demonstrates a significant decrease of the calcium influx is observed in the recombinant cells in presence of said compound compared to the results obtained for the first reference control.

It also preferred a method for selecting or identifying a positive modulator of an ion-channels receptor, said method comprising the following steps of:
A) selecting or identifying a compound capable of modulating the activity of said ion-channels receptor by a method for selecting or identifying a compound capable of modulating the activity of an ion-channels receptor according to the present invention wherein in step b) said recombinant cells are contacted with the compound to be tested in presence of an agonist of said P2X receptor, such as ATP, and the agonist of said ion-channels receptor; and
B) the selection of said compound as a posive modulator if the comparison carried out in step d) of the method according to the present invention demonstrates a significant decrease of the calcium influx is observed in the recombinant cells in presence of said compound compared to the results obtained for the first reference control.

In a more preferred embodiment, in the method for selecting or identifying a posive modulator of an ion-channels receptor according to the present invention, the agonist of the ion-channels receptor is used at a non-saturated concentration in step b) of the method for selecting or identifying a compound capable of modulating the activity of an ion-channels receptor according to the present invention.

In a third aspect, the present invention relates to a method for selecting or identifying a negative modulator, an inhibitor or an antagonist of an ion-channels receptor, said method comprising the following steps of:
A) selecting or identifying a compound capable of modulating the activity of said ion-channels receptor by a method for selecting or identifying a compound capable of modulating the activity of an ion-channels receptor according to the present invention wherein in step b) said recombinant cells are contacted with the compound to be tested in presence of an agonist of said P2X receptor, such as ATP, and the agonist of said ion-channels receptor; and
B) selecting said compound as a positive modulator if the comparison carried out in step d) of the method according to the present invention demonstrates a significant increase of the calcium influx is observed in the recombinant cells in presence of said compound compared to the results obtained for the first reference control.

In a fourth aspect, the present invention is also directed to a method for selecting or identifying a compound capable of modulating the activity of an ion-channels receptor, an agonist a positive or negative modulator, according to the present invention wherein the same recombinants cells used for testing a first compound can be used for testing at least a second compound to be tested. Indeed, the responses of the P2X2 receptors to its agonist, such as ATP, are stable during application (they do not desensitise) and over time (good recovery). It can therefore be used to test many compounds rapidly using one single cell.

In a fifth aspect, the present invention concerns a kit for the selection of a compound capable of modulating the activity of an ion-channels receptor, wherein said kit comprises:
recombinant cells capable of co-expressing an ion-channels receptor and a P2X receptor, or, optionally, means to obtain such recombinant cells;
ATP or any of other P2XR agonist such as α,βmATP (alphabeta-methylene ATP), benzoylbenzoic ATP (such as 2′ and 3′ mixed isomers or 2′,3′-O-(4-benzoylbenzoyl)-ATP (BzATP)), or 2-methylthio-ATP (2-MeSATP);
optionally, an agonist of said ion-channels receptor, such as GABA;
optionally, a calcium probe, preferably a fluorescent calcium probe, as a marker for measuring the calcium influx in said recombinant cells; and
optionally, a system to measure fluorescence such as Video microscopy or Fluorescence Image Plate Recorder (FCIPR) assay.

In the kit according to the present invention, said ion-channels receptor, P2X receptor, recombinant cells and calcium probe are independently chosen among those as defined above for the method of the invention for selecting or identifying a compound capable of modulating the activity of an ion-channels receptor.

The present invention is also directed to a device or system, such system which can be used by high-throughput screening (HTS) for the selection of a compound capable of modulating the activity of an ion-channels receptor, wherein said device comprises the essential elements as defined above for the method or the kit of the invention for selecting a compound capable of modulating the activity of an ion-channels receptor.

This invention can be used to select compounds capable of activating, modulating or inhibiting ion-channels receptor, such as GABA receptors, functionally coupled with P2X receptor, by measuring calcium indicators, particularly with a fluorescent calcium probe capable of measured the calcium influx via the P2X receptor.

In a final aspect, the present invention comprises new compound or compound selected or identified by the methods for selecting or for identifying according to the invention as defined above, these new compounds or already known compounds being newly identified as modulator, such as agonist, positive or negative modulator, of the activity of an ion-channels receptor, said ion-channels receptor being preferably a receptor capable of interacting functionally with P2X receptors, such as GABA, glycine, serotonin and acetylcholine receptors.

In a particular embodiment, said new compounds or already known compounds are compounds being newly identified as modulator, pos of the activity of GABA receptor, particularly the GABA-A receptor, and are selected or identified by the method according to the present invention, wherein in said method, said ion-channels receptor is a GABA receptor, particularly the GABA-A receptor.

The present invention further relates to use of a said compound according to the present invention, for the diagnosis, prevention and treatment of diseases and disorders in mammals, including man, which are related to ion-channels receptor which can interact functionally with P2X receptors.

Preferably, the invention comprises the use of a compound according to the invention for the prevention or the treatment, or for the manufacture of a medicament for the prevention or the treatment, of diseases, disorders or condition in mammals, including man, which are related to ion-channels receptor which can interact functionally with P2X receptors. More preferably, said disease, disorder or condition is related to GABA-A receptor dysfunction.

When said disease, disorder or condition is related to GABA receptor dysfunction, particularly GABA-A dysfunction, preferred are a disease, disorder or condition selected from the group consisting of asthma, acute heart failure, hypotension, urinary retention, osteoporosis, hypertension, angina pectoris, myocardial infarction, ulcers, allergies, benign prostatic hypertrophy, prostate cancer, Parkinson's disease, psychotic and neurological disorders, anxiety, schizophrenia, mania, depression, dyskinesia, memory disorders, sleep disorders, convulsive disorders, or epilepsy.

New compounds identified using the present invention will be useful in the diagnosis, prevention and treatment of diseases and disorders in mammals, including man, Concerning particularly the GABA receptor, these new compounds identified using the present invention will be useful in the diagnosis, prevention and treatment of diseases and disorders such as dysfunction: asthma, hypotension, hypertension, urinary retention, angina, myocardial infarction, ulcers, allergies, Parkinson's disease, neurological disorders, anxiety, schizophrenia, depression, dyskinesia, cognitive disorders, sleep disorders, convulsive disorders and epilepsy.

A test compound identified using the methods of the invention, or a pharmaceutically acceptable salt thereof, is administered to a patient, preferably a mammal, more preferably a human, suffering from a disease whose progression is associated with a lack or a too much permeability of the ion-channels formed by the ion-channels receptor which can interact functionally with P2X receptors, such as GABA, glycine, serotonin and acetylcholine receptors. As used therein, “treatment” refers to an amelioration of a disease, or at least one discernible symptom thereof or to an amelioration of at least one measurable physical parameter, not necessarily discernible by the patient or to delaying the onset of the disease. As used herein, “prevention” refers to a reduction of the risk of acquiring a disease. According to this embodiment, the patient can have a genetic predisposition to a disease, such as a family history of the disease, or a non-genetic predisposition to the disease.

When administered to a patient, a test compound or a pharmaceutically acceptable salt thereof is preferably administered as component of a composition that optionally comprises a pharmaceutically acceptable vehicle. The composition can be administered orally, or by any other convenient route, and may be administered together with another biologically active agent. Administration can be systemic or local. Various delivery systems are known, e.g., encapsulation in liposomes, microparticles, microcapsules, capsules, etc., and can be used to administer the selected compound of the present invention or pharmaceutically acceptable salts thereof.

Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically. The mode of administration is left to the discretion of the practitioner. In most instances, administration will result in the release of a test compound or a pharmaceutically acceptable salt thereof into the bloodstream.

Compositions comprising a test compound selected by the methods according to the present invention, or a pharmaceutically acceptable salt thereof, which form also part of the present invention, can additionally comprise a suitable amount of a pharmaceutically acceptable vehicle so as to provide the form for proper administration to the patient. The term “pharmaceutically acceptable” means approved by a regulatory agency or listed by a national or a recognized pharmacopeia for use in animals, mammals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gelatin, starch and the like. In addition, auxiliary, stabilizing, thickening, lubricating and coloring agents may be used. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, sodium stearate, glycerol monostearate, sodium chloride, dried skim milk, glycerol, propylene, glycol, water and the like. Test compound compositions, if desired, can also contain minor amounts of wetting or emulsifying agents, or pH buffering agents. The compositions of the invention comprising the selected compound can take the form of solutions, suspensions, emulsion, tablets, pills, pellets, capsules, capsules containing liquids, powders, sustained-release formulations, suppositories, emulsions, aerosols, sprays, suspensions, or any other form suitable for use. Said composition is generally formulated in accordance with routine procedures as a pharmaceutical composition adapted to human beings for oral administration or for intravenous administration. The amount of the selected compound or a pharmaceutically acceptable salt thereof that will be effective in the treatment of a particular disease will depend on the nature of the disease, and can be determined by standard clinical techniques. In addition, in vitro or in vivo assays may optionally be employed to help identify optimal dosage ranges. The precise dose to be employed will also depend on the route of administration, and the seriousness of the disease, and should be decided according to the judgment of the practitioner and each patient's circumstances. However, suitable dosage ranges for oral, intranasal, intradermal or intraveneous administration are generally about 0.01 milligram to about 75 milligrams per kilogram body weight per day, more preferably about 0.5 milligram to 5 milligrams per kilogram body weight per day.

Other characteristics and advantages of the invention appear in the continuation of the description with the examples and the figures whose legends are represented below.

### BRIEF DESCRIPTION OF THE FIGURES

FIG. 1: Fluorescence intensity measured after application of ATP, or GABA onto an oocyte not expressing P2X and GABA receptors.

FIG. 2: Fluorescence intensity measured after application of ATP+GABA, GABA or ATP, onto an oocyte expressing the P2X2 receptor.

FIG. 3: Fluorescence intensity measured after application of ATP+GABA, ATP or GABA, onto an oocyte co-expressing the GABA-A receptor and P2X2 receptor.

FIG. 4: Fluorescence intensity measured after application of GABA at different moment of the ATP response, onto an oocyte co-expressing the GABA-A receptor and P2X2 receptor.

### EXAMPLE

### Example 1

### Material and Methods

Using an automatic nano injector, cDNA encoding rat P2X2 receptors and/or GABA receptors composed for example of rat a2 and rat b3 subunits have been injected into the nucleus of Xenopus oocytes as previously described in Boué-Grabot et al., 2004 (Journal of Biological Chemistry, 279; 52517-52525, 2004).

1 to 3 days after infection, the calcium green fluorescence probe, which is sensitive to the concentration of free intracellular calcium, was injected into oocytes expressing either P2X2 receptors alone, P2X and GABA receptors or no receptors (not injected). Then, using video-microscopy (Nikon), the fluorescence emitted by oocytes maintained in a standard solution (Ringer solution containing 1.8 mM calcium chloride), perfused at 3 ml/min, has been recorded. The substances (ATP 100 mM, GABA 100 μM, ATP+GABA 100 μM of each) diluted in the same buffer were applied using an application system (BPS8-AlaScientific). Variations in fluorescence were analysed using IpLab software.

### Example 2

### Oocytes Expressing No Receptors

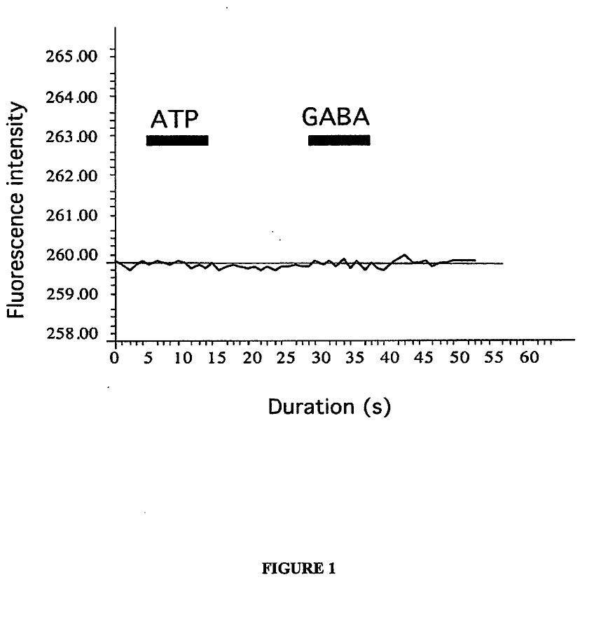

No modification of the fluorescence was observed on application of ATP or GABA onto an oocyte not expressing P2X and GABA receptors (FIG. 1). These experiments confirm that the oocytes do not express endogenous receptors that are sensitive to ATP or GABA.

### Example 3

### Recording from Oocytes Co-Expressing P2X2 Receptors Alone

The application of ATP induces a transient increase in fluorescence that demonstrates the influx of calcium due to opening of the P2X2 receptor channels. GABA had no effect on the fluorescence, indicating that GABA does not activate P2X2 receptors. The co-application of ATP+GABA also results in an increase in fluorescence similar to that obtained with ATP alone.

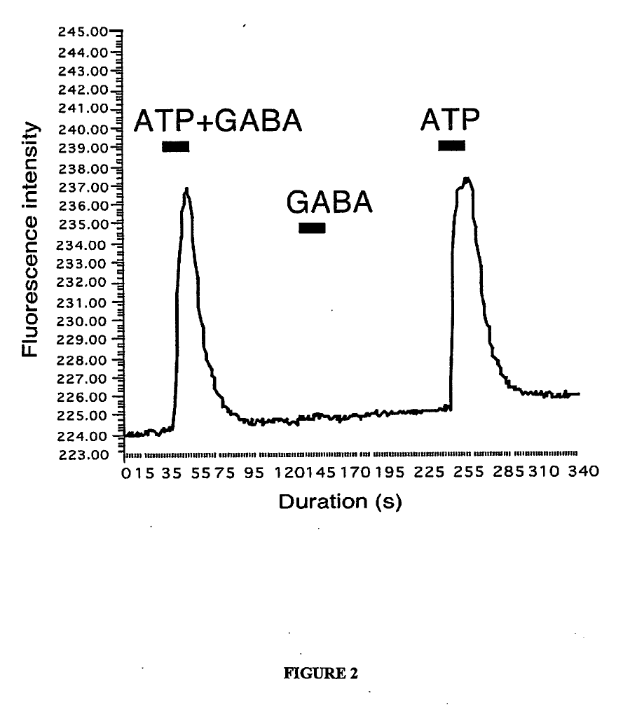

The increase in fluorescence induced by application of ATP onto oocytes expressing only P2X2 receptors is not altered by GABA (see FIG. 2).

### Example 4

### Recording of Oocytes Co-Expressing P2X2 and GABA-A Receptors

An application of ATP (100 μM) induces a marked transient increase in the fluorescence of the calcium probe, demonstrating activation of the P2X receptors and the subsequent influx of calcium (FIG. 2). Successive applications of ATP induce responses of similar amplitude. It is important to note that this increase in fluorescence is observed at the cell's resting potential.

Application of GABA alone (100 μM) does not modify fluorescence intensity. This can be explained by the fact that activation of the GABA receptors causes a chloride channel to open, which has no effect on intracellular calcium concentration.

If a solution of ATP+GABA (100 μM of each) is co-applied, we obtain a lower calcium response than that obtained by applying ATP alone, thus showing that the opening of the GABA channels inhibits the calcium influx mediated by P2X receptors.

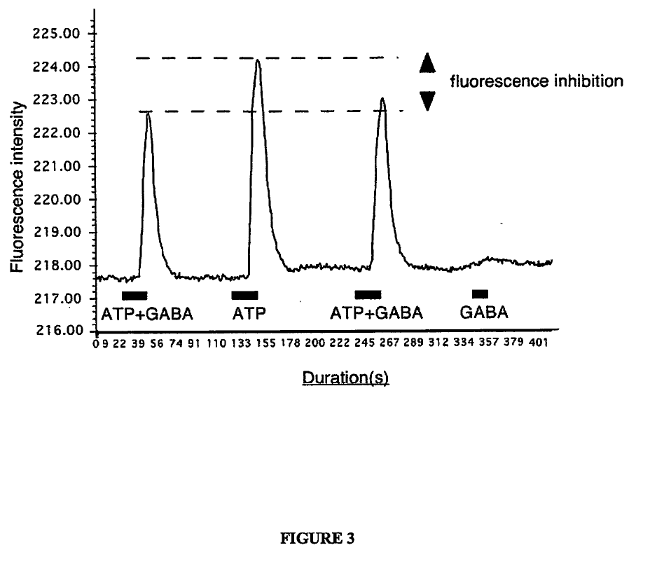

This decrease in fluorescence shows the activation of the GABA-A receptors (FIG. 3).

The percentage inhibition obtained (20-25% in these experiments) is a reflection of the expression ratio of the P2X and GABA receptors. As we have shown using electrophysiological techniques, if the ratio of P2X2 to GABA receptors is 1:1, the current inhibition by GABA of the response mediated by ATP can then reach 50%. By calcium imaging, only opening of P2X channels is measured. So it can be postulate that if GABA-A receptors interact with expressed P2X2 receptors, activation of GABA receptors could lead to the closure of all P2X receptor.

### Example 5

### Recording of the Activation or Inhibition of GABA Receptors During the Activation of P2X Receptors of Oocytes Co-Expressing P2X2 and GABA-A Receptors

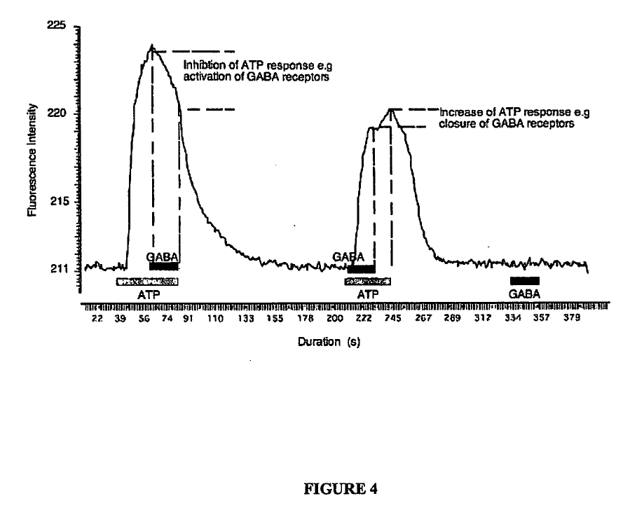

The activation or inhibition of GABA receptors can also be monitored during the activation of P2X receptors (see FIG. 4).

If GABA is applied at the peak of the ATP response, a decrease in fluorescence intensity is observed, showing that when the GABA receptors are opened it leads to closure of the P2X receptors that were already open. And likewise, if during co-application of ATP and GABA, the application of GABA is stopped, an increase in fluorescence is observed. This indicates that closure of the GABA receptors lifts the inhibition of some of the P2X receptors, i.e. allows the P2X receptors to open (FIG. 4).

This shows that irrespective of the sequence in which the agonists are applied, opening or closure of the GABA-A receptors directly modulates the calcium current (measured by the fluorescence of the calcium-sensitive probe) of the P2X channels induced by ATP. The measurement of calcium influx from the P2X receptors therefore represents a new procedure to monitor the activity of GABA receptors.

These experiments show the co-expression of rat GABA-A alpha2 beta3 and P2X2 receptors, but the present invention can be used to screen all rat, mouse or human GABA-A receptors using the P2X2 receptor or any other wild-type or mutated P2X receptors that retain the ability to interact with GABA receptors.

1. A method for selecting or identifying a compound capable of modulating the activity of an ion-channels receptor, wherein said method comprises the following steps of:
a) having recombinant cells expressing said ion-channels receptor and a P2X receptor;
b) contacting said recombinant cells with the compound to be tested in presence of an agonist of said P2X receptor, or in presence of an agonist of said P2X receptor and the agonist of said ion-channels receptor;
c) measuring the calcium influx via the P2X receptor;
d) comparing the measure obtained at step c) with the measure obtained in the same conditions for a first reference control wherein in step b) the compound to be tested is not present; and
e) selecting said compound if the comparison in step d) demonstrates a significantly increase or decrease of the calcium influx in the recombinant cells in presence of said compound.

2. The method according to claim 1, wherein at step a) said recombinant cells do not express endogenously a receptor capable of being modulated by said agonist of said P2X receptor or by the agonist of said ion-channels receptor.

3. The method according to claim 1, wherein said recombinant cells in step a) are mammal cells or Xenopus oocytes.

4. The method according to claim 1, wherein said P2X receptor is a natural P2X receptor or a mutated P2X receptor capable of interacting with said ion-channels receptor.

5. The method according to claim 4, wherein said P2X receptor is the P2X1, P2X2, P2X3, P2X4, P2X5, P2X6 or P2X7 receptor or any P2X receptor which results from the association or the combination of P2X subunit receptor capable of exercising an ATP activated channel-receptor function.

6. The method according to claim 1, wherein said ion-channels receptor or said P2X receptor is from human, rat or mouse source.

7. The method according to claim 1, wherein said agonist of said P2X receptor is selected from the group consisting of ATP, α,β mATP (alphabeta-methylene ATP), benzoylbenzoic ATP, and or 2-methylthio-ATP (2-MeSATP).

8. The method according to claim 1, wherein at step c) the calcium influx is measured by a calcium probe.

9. The method according to claim 8, wherein the calcium infux is measured by a fluorescent calcium probe, wherein said fluorescent calcium probe is calcium green, Fluo-3 or Fluo-4.

10. The method according to claim 1, wherein said compound to be tested is tested for its ability to interact specifically with said P2X receptor, wherein said specific interacting is demonstrated by measuring whether a significant increase or decrease of the calcium influx is obtained in the same conditions for a second reference control wherein in step a) the recombinant cells used for said second reference control express said P2X receptor and do not express said ion-channels receptor.

11. The method according to claim 10, wherein said compound to be tested is not selected if a significant increase or decrease of the calcium influx is observed in the recombinant cells for said second reference control.

12. A method for selecting or identifying an agonist of an ion-channels receptor, said method comprising the following steps of:
A) selecting or identifying a compound capable of modulating the activity of said ion-channels receptor by the method according to claim 1 wherein in step b) said recombinant cells are contacted with the compound to be tested only in presence of an agonist of said P2X receptor; and
B) the selection of said compound as an agonist if the comparison carried out in step d) of the method according to claim 1 demonstrates a significant decrease of the calcium influx is observed in the recombinant cells in presence of said compound compared to the results obtained for the first reference control.

13. A method for selecting a positive modulator of an ion-channels receptor, said method comprising the following steps of:
A) selecting or identifying a compound capable of modulating the activity of said ion-channels receptor by the method according to claim 1 wherein in step b) said recombinant cells are contacted with the compound to be tested in presence of an agonist of said P2X receptor and the agonist of said ion-channels receptor; and
B) the selection of said compound as a positive modulator if the comparison carried out in step d) of the method according to claim 1 demonstrates a significant decrease of the calcium influx is observed in the recombinant cells in presence of said compound compared to the results obtained for the first reference control.

14. A method for selecting a positive modulator of an ion-channels receptor according to claim 13, wherein in step b) of the method according to claim 1, in A), the agonist of said ion-channels receptor is used at a non-saturated concentration.

15. A method for selecting or identifying a negative modulator, an inhibitor or an antagonist of an ion-channels receptor, said method comprising the following steps of:
A) selecting a compound capable of modulating the activity of said ion-channels receptor by a method according to claim 1 wherein in step b) said recombinant cells are contacted with the compound to be tested in presence of an agonist of said P2X receptor and the agonist of said ion-channels receptor; and
B) the selection of said compound as a positive modulator if the comparison carried out in step d) of the method according to claim 1 demonstrates a significant increase of the calcium influx is observed in the recombinant cells in presence of said compound compared to the results obtained for the first reference control.

16. The method according to claim 1, wherein the same recombinant cells used for testing a first compound are used for testing at least a second compound to be tested.

17. The method according to claim 1, wherein said ion-channels receptor is the GABA receptor, the glycine receptor, the acetylcholin receptor or the serotonin receptor.

18. The method according to claim 17, wherein said ion-channels receptor is the GABA receptor.

19. The method according to claim 17, wherein said ion-channels receptor is a ion-channels GABA receptor resulting from the homomeric or heteromeric association between any GABA receptor subunit, wherein said subunit is selected from the group of GABA receptor subunits consisting of alpha 1 to 6, beta 1 to 3, gamma 1 to 3, delta, epsilon, theta, pi, and rho 1 to 3 subunit, and wherein said homomeric or heteromeric association forms an ion-channels receptor capable of being activated by GABA.

20. The method according to claim 18, wherein the agonist of said ion-channels receptor is GABA or a GABA receptor agonist.

21. A kit for the selection of a compound capable of modulating the activity of an ion-channels receptor, wherein said kit comprises:
a) recombinant cells capable of co-expressing an ion-channels receptor and a P2X receptor;
b) an agonist of P2X receptor selected from the group consisting of ATP, α,β mATP (alphabeta-methylene ATP), benzoylbenzoic ATP, and 2-methylthio-ATP (2-MeSATP);
c) an agonist of said ion-channels receptor;
d) a calcium probe as a marker for measuring the calcium influx in said recombinant cells.

22. The kit according to claim 20, wherein said ion-channels receptor is a GABA receptor, a glycine receptor, an acetylcholine receptor, or a serotonin receptor, wherein said P2X receptor is a natural P2X receptor or a mutated P2X receptor capable of interacting with said ion-channels receptor, wherein said recombinant cells do not express endogenously a receptor capable of being modulated by said agonist of said P2X receptor or by the agonist of said ion-channels receptor, and wherein said calcium probe is a fluorescent calcium probe.

23. (canceled)

24. A compound selected or identified by a method according to claim 1.

25. The compound according to claim 24, wherein in said method, said ion-channels receptor is a GABA receptor.

26. A method for the diagnosis, prevention, or treatment of a disease or disorder in mammals which are related to ion-channels receptors that interact functionally with P2X receptors comprising administering to said mammal the compound according to claim 24.

27. The method according to claim 26, wherein said mammal is man.

28. The method according to claim 26, wherein said disease or disorder is related to GABA-A receptor dysfunction.

29. The method according to claim 28, wherein said disease or disorder is asthma, acute heart failure, hypotension, urinary retention, osteoporosis, hypertension, angina pectoris, myocardial infarction, ulcers, allergies, benign prostatic hypertrophy, prostate cancer, Parkinson's disease, psychotic and neurological disorders, anxiety, schizophrenia, mania, depression, dyskinesia, memory disorders, sleep disorders, convulsive disorders, or epilepsy.
